(19) United States
(12) Patent Application Publication
(10) Pub. No.: US 2015/0342514 A1
(43) Pub. Date: Dec. 3, 2015

(54) SYSTEM AND METHOD FOR MULTIPLE ALLERGEN TESTING

(71) Applicant: Kenneth Easton, Palm Beach, FL (US)

(72) Inventor: Kenneth Easton, Palm Beach, FL (US)

(21) Appl. No.: 14/289,709

(22) Filed: May 29, 2014

(51) Int. Cl. A61B 5/00

(52) U.S. Cl. CPC A61B 5/411

(57) ABSTRACT

The testing apparatus includes a capsule 108 tray and a hand held allergen applicator 104 that facilitates convenient storage and easy application of such allergens simultaneously. The hand held applicator 104 includes a plurality of prongs to administer the respective plurality allergen under the skin of the patient. The allergy testing apparatus provides for efficient pre-wetting of the prongs with respective allergens to save the time that would otherwise be required to apply the respective allergens to each of the plurality of prongs immediately before performing an allergy test. In addition, the apparatus provides for efficient use of the allergen testing materials thereby reducing material costs while limiting the amount of residual, unused allergen.

### BACKGROUND OF THE INVENTION

1. Field of the Invention

Embodiments of the claimed invention generally relates to systems and methods for testing medical conditions and, more particularly, to systems used to determine a degree of reaction to one or more allergens by a subject in a minimally invasive manner.

2. Description of the Related Art

Skin testing to identify potential allergens that a patient may be allergic to is capsule 108 known in many forms. The person conducting the skin testing may apply a relatively large number of test solutions to a patient's skin. To perform skin tests of this type, the person conducting the skin test typically places the skin test devices into a test solution such that a small amount of test solution adheres to the sharp pointed ends on the legs of the skin testing device via capillary attraction. The loaded skin test device is then pressed into a site on the patient's skin in a predetermined sequence. However, the sharp pointed ends of the skin test device may cause discomfort to some patients and much pain to other patients because the sharp pointed ends must penetrate the epidermis of the skin in order to get meaningful allergy test results.

Physicians prescribe allergy testing because early diagnosis can help patients take preventive measures such as managing their environment, avoiding certain drugs or foods, availing themselves of medication, immunotherapy, or self-administered epinephrine. Allergy tests such as hypersensitivity skin testing have been used for decades because of their sensitivity, reliability, and relative immediacy of results. The widely-used Skin Prick (percutaneous) Test involves the manual application of as many as 40 standardized allergen extracts in a labeled array pattern on the patient's back or forearm, subsequently pricking the region with a needle to allow diffusion of allergens into the patient's epidermal tissue. The intradermal test (intracutaneous) is somewhat more invasive and labor intensive since it involves injecting small amounts of allergens into the patient's skin. The procedure, while more sensitive than percutaneous tests, has a somewhat lower predictive value. Intracutaneous testing is most often used to diagnose insect venom and drug allergies. Due to the nature of these processes, large areas of the subject's skin tend to be affected.

In both of these types of hypersensitivity skin testing, reactions to the allergens are assessed after a time period of approximately 20 minutes based on the area and coloration of wheals or erythemas produced by each allergen. Sensitivity is diagnosed and recorded using semi-quantitative guidelines and controls such as saline and histamine. Although the procedure is not highly painful and produces only temporary tenderness, itching, or swelling, it can lead to significant stress and anxiety in many patients, especially children. In addition to psychological effects the test may have on some patients, there may be other related problems: 1) Degree of invasiveness and skin surface area covered; 2) Time and manual labor needed to label, dispense, and prick regions with allergens; 3) Lack of an automated standardized protocol to capture the data and store as a digital record.

Blood testing provides an invasive, yet possibly more convenient method of allergy testing. Unfortunately, blood testing does not surpass the sensitivity, specificity, and predictive value of the skin test. Blood test results are often dependent upon the laboratory which is performing the test. Furthermore, blood testing for allergies is also a more expensive option.

The alarming prevalence of allergies and their rapid growth indicate that the demand for allergists and immunologists will increase in the years to come. Unfortunately, American Academy of Allergy, Asthma, & Immunology (AAAAI) surveys indicate that the number of graduating allergists may not be sufficient to address future health needs. Furthermore, demographic studies of the approximately 4000 allergists in the U.S. show that a disproportionately high number of core allergists will likely retire in the near future, further exacerbating the problem. These trends indicate not only that the number of allergists per capita will decline but, more importantly, that the number of allergists per allergy patient will decline at an even faster rate. Not surprisingly, allergy practices, especially those run by younger physicians, are experiencing rapid growth in the number of patient visits.

These trends point to a compelling need for less invasive, more quantitatively productive allergy tests with shorter procedure times. Less invasive, shorter, lower cost procedures are also likely to reduce patient fear and reduce barriers associated with current procedures. Improved testing might also promote early or preventative allergy screening mitigating “allergic march”, sensitization, or the cost and trauma of serious reactions.

Therefore, there is a need for a minimally invasive allergy testing system which does not need to cover large areas of a patient's skin, which can be correlated to existing skin testing data, which is economical, and which is easy to use and manufacture.

### SUMMARY

In accordance with one embodiment, an allergy testing kit 100 for applying more than one allergens to a cutaneous site on a patient. The allergy testing kit 100 may have a Base member 102 having a top side and a bottom side. The Base member 102 may have more than one openings.

The top side of the Base member 102 comprising a recessed depression concentric to each of the plurality of the openings, wherein the recessed depression having a diameter larger than the opening. In one embodiment, the allergy testing kit 100 comprises more than one capsule 108 configured to be received in each of the plurality of the openings within the Base member 102. The plurality of capsule 108 having a capsule 108 portion, the capsule 108 portion holding a predetermined amount of biologically active allergen.

The plurality of capsule 108 having an opening at the opposite ends of the well. The capsule 108 opening may have a rim on the outer edge of the capsule 108 opening. The capsule 108 rim is adapted for insertion into the recessed depression of the Base member 102. The allergy testing kit 100 may further have a top cover 106 member configured to fasten with the Base member 102.

The allergy testing kit 100 further includes an applicator 104 having a plurality of circular flat plates (hereinafter “guard 110s”). Furthermore, the test kit 100 may have more than one latitudinal ridges (“beam 112s”) having the guard 110 at each end of the beam 112. A single upwardly extending latitude rigid (“grip 114”) adapted for manually grasping the device is provided. The grip 114 attached to the plurality of beam 112s at opposing edge to the guard 110s. The allergy kit 100 comprises more than one downwardly extending prongs each prong connected to at bottom of the guard 110. Each of the plurality of prongs adapted for insertion into the corresponding Base member 102 opening.

The prong comprises a first side and a second side; the first side comprises a brush for capturing liquid from the capsule 108 via capillary attraction, the second side connected to the guard 110. Each of the prongs having a predetermined fixed length. The guard 110 adapted to fit in the Base member 102's recessed depression. The allergy testing kit 100 comprises more than one triangular recessed depression to orient an applicator 104, wherein the apex of the triangle, opposite to the base of the triangle, corresponding to the front end of the applicator 104.

For example, the allergy testing kit 100 wherein the eight of the plurality of openings are grouped in two columns with of four openings in each column. The allergy testing kit 100 with the base having a triangular recessed depression is placed between the first two openings. The applicator 104 also comprises a triangular portion having an apex and a base, the base attached to the front of the outermost beam 112 of the applicator 104. The triangular portion of the applicator 104 having a corresponding triangular recessed depression in the Base member 102, wherein the triangular portion fits into the triangular recessed depression. The applicator 104 having four beam 112s with eight prongs.

The allergy testing kit 100 having more than one capsule 108 shells, wherein each of the plurality of capsule 108 shells configured to receive the capsule 108s containing the biologically active allergen via the opening in the Base member 102. The allergy testing kit 100 having sufficient quantity of biologically active allergen for up to thirty allergy tests. The allergy testing kit 100 further comprises enforcement bars attached to the grip 114. The allergy testing kit 100 wherein the grip 114 is a hollow ridge.

The allergy testing kit 100 wherein the top cover 106 member having a first latch placed at the center of the width at the first end and a second latch placed at the center of the width at the second end. The allergy testing kit 100 wherein the Base member 102 having a catch mechanics to enable attachment of the top cover 106 member. The allergy testing kit 100 wherein the brush is made with multiple abrading elements.

### BRIEF DESCRIPTION OF THE DRAWINGS

The drawings, in which like numerals represent similar parts, illustrate generally, by way of example, but not by way of limitation, various embodiments discussed in the present document.

FIG. 1 illustrates an allergy testing kit 100 assembly in accordance with an embodiment.

FIG. 2 illustrates side profile view of the allergy testing kit 100 assembly in accordance with an embodiment.

FIG. 3 illustrates a side sectional view of one set of allergy testing kit 100 capsule 108s and an applicator 104 in accordance with an embodiment.

FIG. 4 illustrates a top profile view of an applicator 104 in accordance with an embodiment.

FIG. 5 illustrates a bottom view of the applicator 104 in accordance with an embodiment.

FIG. 6 illustrates a profile view of the allergy testing kit 100 assembly without the applicator 104 in accordance with an embodiment.

### DETAILED DESCRIPTION

The foregoing summary, as capsule 108 as the following detailed description of certain embodiments of the subject matter set forth herein, will be better understood when read in conjunction with the appended drawings. As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or more than one elements having a particular property may include additional such elements not having that property.

To the extent that the term “includes” is used in either the detailed description or the claims, such term is intended to be inclusive in a manner similar to the term “comprising” as “comprising” is interpreted when employed as a transitional word in a claim. Furthermore, the term “or” as used in either the detailed description or the claims is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B.

In the following detailed description, reference is made to the accompanying drawings which form a part hereof, and in which are shown by way of illustration specific embodiments in which the subject matter disclosed herein may be practiced. These embodiments, which are also referred to herein as “examples,” are described in sufficient detail to enable those skilled in the art to practice the subject matter disclosed herein. It is to be understood that the embodiments may be combined or that other embodiments may be utilized, and that structural, logical, and electrical variations may be made without departing from the scope of the subject matter disclosed herein. The following detailed description is, therefore, not to be taken in a limiting sense, and the scope of the subject matter disclosed herein is defined by the appended claims and their equivalents. In the description that follows, like numerals or reference designators will be used to refer to like parts or elements throughout. In this document, the terms “a” or “an” are used, as is common in patent documents, to include one or more than one. In this document, the term “or” is used to refer to a nonexclusive or, unless otherwise indicated. Furthermore, references to “one embodiment” are not intended to be interpreted as excluding the existence of additional embodiments that also incorporate the recited features. Moreover, unless explicitly stated to the contrary, embodiments “comprising” or “having” an element or more than one elements having a particular property may include additional such elements not having that property.

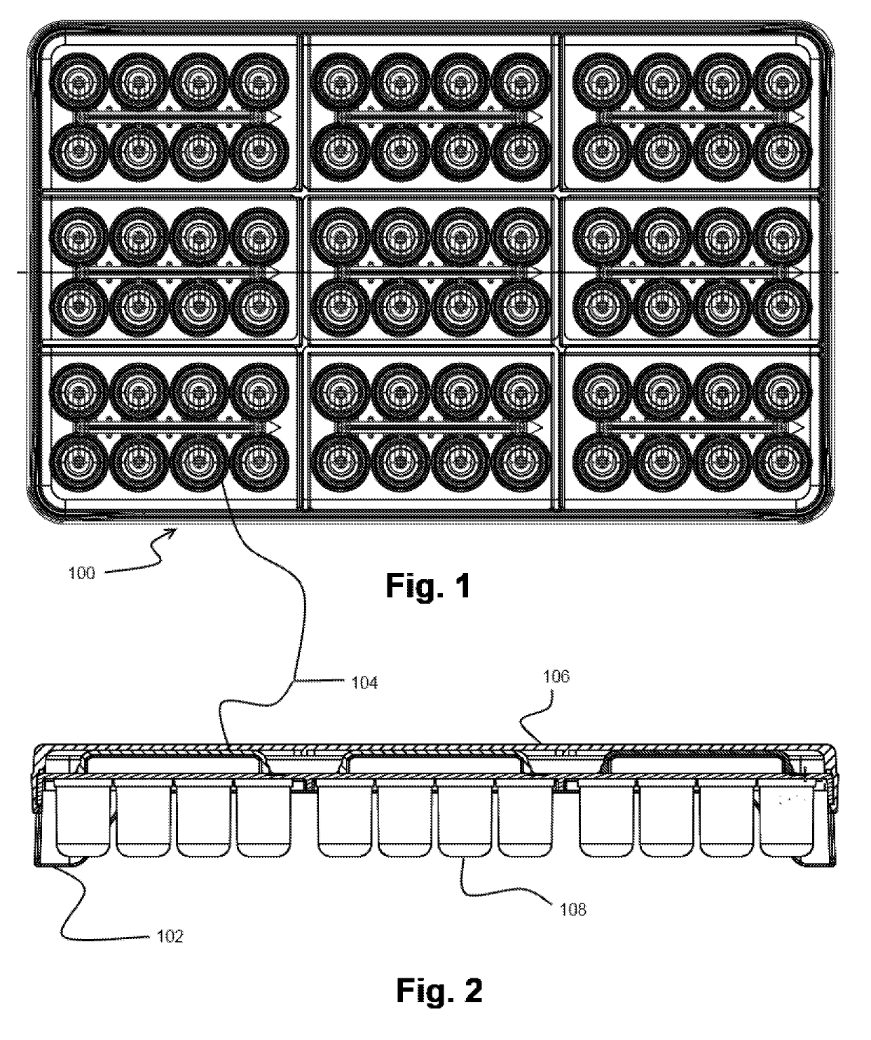

FIG. 1 illustrates an allergy testing kit 100 assembly in accordance with an embodiment. In an exemplary embodiment, an allergy testing system is disclosed. The allergy testing system comprises a skin test device and more than one wells, each having a reservoir for receiving test solutions such as potential allergens or controls. Optionally, each of the capsule 108 may be sealably prepackaged with biologically active allergen having a penantrable screen covering the opening. The allergy testing kit 100 may have a Base member 102 having a top side and a bottom side. The Base member 102 may have more than one openings.

The top side of the Base member 102 comprising a recessed depression concentric to each of the plurality of the openings, wherein the recessed depression having a diameter larger than the opening. In one embodiment, the allergy testing kit 100 comprises more than one capsule 108 configured to be received in each of the plurality of the openings within the Base member 102. The plurality of capsule 108 having a capsule 108 portion, the capsule 108 portion holding a predetermined amount of biologically active allergen.

The plurality of capsule 108 having an opening at the opposite ends of the well. The capsule 108 opening may have a rim on the outer edge of the capsule 108 opening. The capsule 108 rim is adapted for insertion into the recessed depression of the Base member 102. The allergy testing kit 100 may further have a top cover 106 member configured to fasten with the Base member 102.

More than one legs extends from the grip 114, and the plurality of legs is spaced and oriented to contact a corresponding capsule 108 containing the test solution. Each of the plurality of legs has a test head, and each test head has more than one elongated spike members. The elongated spike members have sharp ends configured to receive the test solution or potential allergen from a well, for example via capillary attraction, and to puncture a patient's skin to deliver the test solution or allergen.

For example, the allergy testing kit 100 wherein the eight of the plurality of openings are grouped in two columns with of four openings in each column. The allergy testing kit 100 with the base having a triangular recessed depression is placed between the first two openings. The applicator 104 also comprises a triangular portion having an apex and a base, the base attached to the front of the outermost beam 112 of the applicator 104. The triangular portion of the applicator 104 having a corresponding triangular recessed depression in the Base member 102, wherein the triangular portion fits into the triangular recessed depression. The applicator 104 having four beam 112s with eight prongs.

The allergy testing kit 100 having more than one capsule 108 shells. The capsule 108 shell is integrally formed of thermoplastic and is generally in the form of a hollow downwardly opening tubular box having an upstanding vertical peripheral wall, the bottom edge thereof terminating in a horizontal plane to define a bottom support for the capsule 108.

Each of the plurality of capsule 108 shells configured to receive the capsule 108s containing the biologically active allergen via the opening in the Base member 102. The allergy testing kit 100 having sufficient quantity of biologically active allergen for up to thirty allergy tests. The allergy testing kit 100 further comprises enforcement bars attached to the grip 114. The allergy testing kit 100 wherein the grip 114 is a hollow ridge.

The allergy testing kit 100 wherein the top cover 106 member having a first latch placed at the center of the width at the first end and a second latch placed at the center of the width at the second end. The allergy testing kit 100 wherein the Base member 102 having a catch mechanics to enable attachment of the top cover 106 member. The allergy testing kit 100 wherein the brush is made with multiple abrading elements.

The allergy testing kit 100 of claim 1, wherein each capsule 108 is sealably prepackaged with biologically active allergen having a penantrable screen covering the opening providing for convenient storage and packaging.

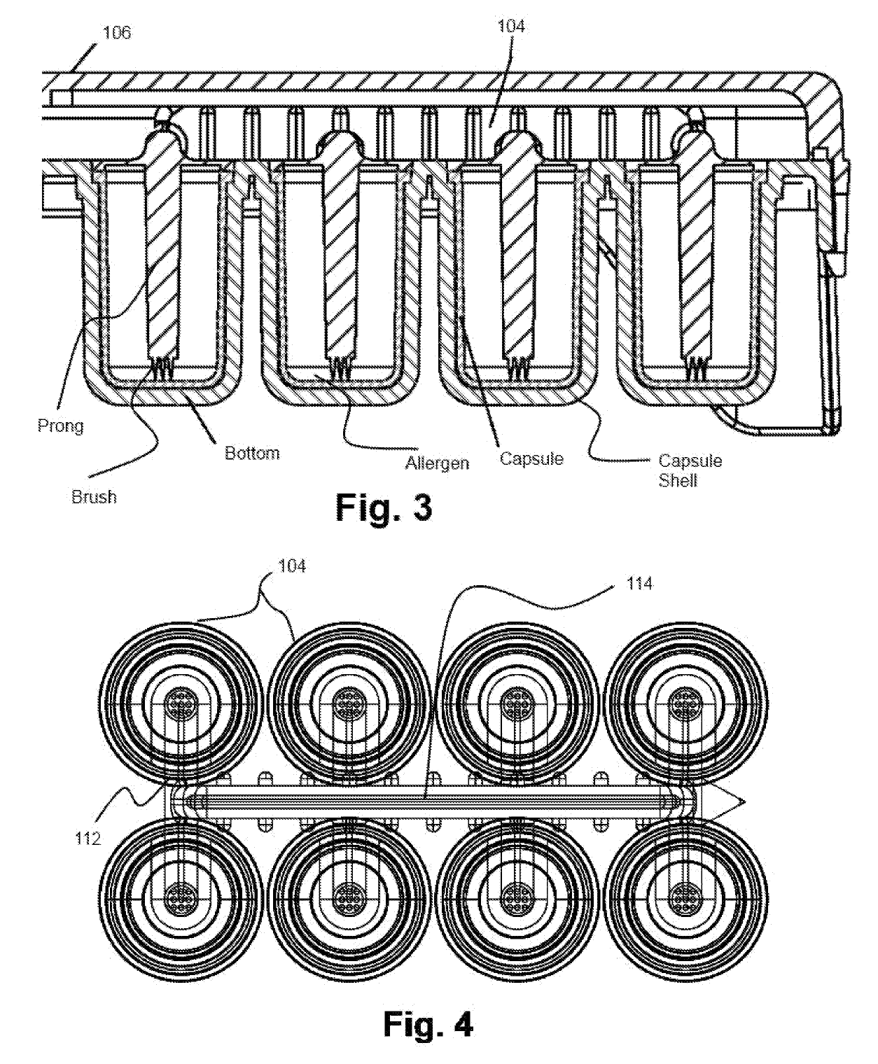

Referring to FIGS. 2 and 3, FIG. 2 illustrates side profile view of the allergy testing kit 100 assembly in accordance with an embodiment. FIG. 3 illustrates a side sectional view of one set of allergy testing kit 100 capsule 108s and an applicator 104 in accordance with an embodiment. In one embodiment, the allergy testing kit 100 is formed of high strength thermoplastic materials such as PET-G or polycarbonate. The peripheral wall is integrally formed with a horizontal web defining a top wall formed integrally with more than one recessed opening configured to accept a capsule 108. The capsule 108 having a well formed in predetermined spaced into which may be disposed therein a predetermined volume of different respective allergens. The openings in Base member 102, for purposes of illustration, are spaced apart in two adjacent parallel rows and configured to receive in their respective open top ends respective prongs of the hand held applicator 104 described hereinafter.

The allergy testing kit 100 further includes an applicator 104 having a plurality of circular flat plates (hereinafter “guard 110s”). Furthermore, the test kit 100 may have more than one latitudinal ridges (“beam 112s”) having the guard 110 at each end of the beam 112. A single upwardly extending latitude rigid (“grip 114”) adapted for manually grasping the device is provided. The grip 114 can have an elongated portion for holding the device. The grip 114 attached to the plurality of beam 112s at opposing edge to the guard 110s. The allergy kit 100 comprises more than one downwardly extending prongs each prong connected to at bottom of the guard 110. Each of the plurality of prongs adapted for insertion into the corresponding Base member 102 opening.

The plurality of downwardly projecting allergen prongs are spaced apart in conformity with the predetermined spaced configuration of the plurality of opening. The lateral opposite sides of the hand held applicator 104 is also integrally formed from a mold during the thermoplastic molding process. The handles are configured to be grasped at their respective top extremities by a user. The grip 114 have such a configuration for ease of handling the applicator 104 as for ease of applying the requisite compressive force to the skin of the patient to without breaking the surface of such skin.

The prong comprises a first side and a second side; the first side comprises a brush for capturing liquid from the capsule 108 via capillary attraction, the second side connected to the guard 110. Each of the prongs having a predetermined fixed length. The guard 110 adapted to fit in the Base member 102's recessed depression.

As shown in FIG. 2, the applicator 104 is configured to cooperate with the capsule 108 such that it may be aligned thereover and positioned in complementary relationship thereon to immerse the respective distal ends of the respective prongs within the respective capsule 108s. The applicator 104 may be releasably removed from the capsule 108 tray for application of the respective allergens to the patient.

With particular reference to FIG. 2, the capsule 108 will be described in more detail. In accordance with an embodiment, the capsule 108 is constructed of lightweight, thin wall, thermoplastic material and may be molded over a forming mold using vacuum forming processes capsule 108 known by those skilled in the art. As such, the peripheral wall of the capsule 108 may be formed with a slight downwardly diverging draft to facilitate removal of the capsule 108 from the Base member 102. The capsule 108s are generally cylindrical having cylindrical open top ends to define respective necks.

As shown in FIG. 2, the bottom ends of the respective capsule 108s convergingly taper in a smooth curved manner radially inwardly and then downwardly to form respective reduced-in-diameter collection basins defining sinks/wells having respective flat bottom surfaces. The respective wells are formed to have uniform, predetermined depths such that the bottom surface of the collection sinks terminate in a common horizontal plane. The wells serve to hold therein the desired quantities of various liquid allergens which are to be applied to a patient undergoing an allergy test.

FIG. 4 illustrates a top profile view of an applicator 104 in accordance with an embodiment. With reference to FIG. 4, the hand held applicator 104 will be described in further detail. In the preferred configuration, the applicator 104 may also be constructed of thermoplastic materials and formed, for instance, by an injection molding processes capsule 108 known by those skilled in the art. Preferably, the hand held applicator 104 is constructed of either polypropylene, acetal, or polycarbonate. Using such a molding process, the prongs are formed integrally at their upper extremities with the guard 110 of the applicator 104. More particularly, the prongs are in the form of elongated stems terminating at their respective bottom extremities in respective penetration points configured to be normally immersed in the respective capsule 108s and formed as a brush.

The bursh may be sharp jagged edges to irritate the skin surface upon application thereto. The stems of the prongs are generally slender and cylindrical, although their particular shape and cross sectional geometry may vary. The respective stems of the prongs are further formed with uniform, predetermined lengths such that the penetration points are disposed in a common plane to be simultaneously pressed against the skin of a patient. In addition, the upper ends of the respective prongs are connected to a guard 110. The guard 110s are enlarged-in-diameter concentric to the prongs forming a plug for the capsule 108 sized for snug receipt within the respective recessed depression in the Base member 102. The guard 110 at the top open ends of the respective capsule 108s help retain the respective allergens stored therein.

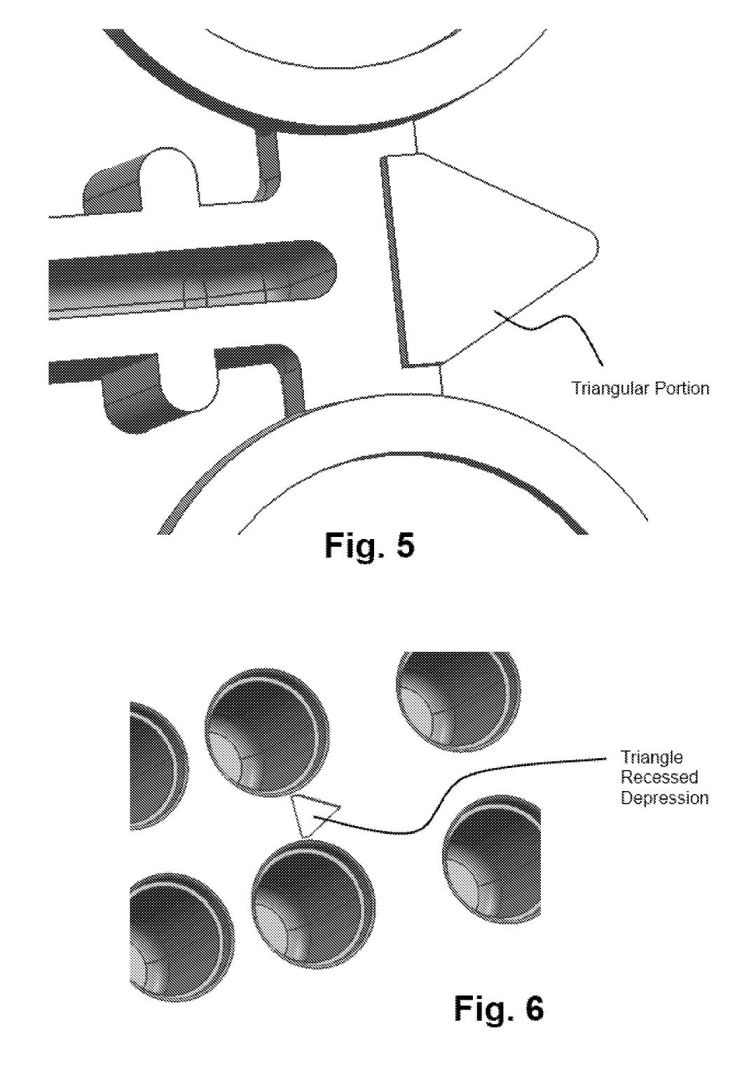

FIG. 5 illustrates a bottom view of the applicator 104 in accordance with an embodiment. FIG. 6 illustrates a profile view of the allergy testing kit 100 assembly without the applicator 104 in accordance with an embodiment. The allergy testing kit 100 comprises more than one triangular recessed depression to orient an applicator 104, wherein the apex of the triangle, opposite to the base of the triangle, corresponding to the front end of the applicator 104. In one embodiment, the triangle on the applicator 104 is configured to snug fit within the triangle recessed depression within the Base member 102. It is to be appreciated that the allergy testing kit 100 is configured to orient the user of the kit 100 as to the positioning of the applicator 104. Thus, when the applicator 104 is applied to the patient, the user will not confuse as to the front side of the applicator 104 as was inserted within the capsule 108.

It is to be appreciated that the allergy testing kit 100 is configured to be conveniently packaged for subsequent sale and distribution to clinicians for performing allergy tests. To assemble and package the allergy testing apparatus, the capsule 108 and hand held applicator 104 may be fabricated as described above and then sterilized using techniques known to those skilled in the art. Thereafter, predetermined volumes of commonly used pre-selected respective allergens are poured into the respective capsule 108s.

Thereafter, the applicator 104 may be positioned over the capsule 108 to align the respective prongs downwardly over the respective capsule 108s and the applicator 104 advanced downwardly thereon to immerse the respective brushes within the respective allergens as the respective guard 110s are received within the respective recessed depression of the Base member 102. The applicator 104 may be advanced downwardly until the guard 110 of the applicator 104 comes to rest on the top of the capsule 108 rim. It is to be appreciated that the length of the stems of the respective prongs and the depth of the respective capsule 108 are so configured to dispose the distal ends of the respective stems in close spaced relationship with, but spaced slightly from, the bottom surface of the respective capsule 108. This positions the brush such that they will be submersed in the respective allergens, even as the volume of such allergens approach depletion while preventing direct engagement with the respective walls of the bottom to thereby maintain the integrity of the brush. It can be appreciated that the fabrication and assembly of the allergy testing kit 100 is completed in a minimum number of steps to minimize production costs.

Furthermore, it is to be appreciated that the respective capsule 108s may be filled with different selected combinations of allergens for use in performing tests for different allergies. As such, a user may select a particular allergy testing apparatus containing a certain combination of allergens to be administered to patients exhibiting particular allergic reactions.

Thereafter, the applicator 104 and capsule 108 may be packaged as by, for instance, vacuum sealing in a protective sterilized plastic cover to package the allergy testing apparatus for storage. In addition, the packaged allergy testing kit 100 have a top cover 106 to further protect the testing apparatus from damage or tampering and to facilitate safe shipping and handling. It is to be appreciated that the respective prongs are thus assembled and packaged for storage to be maintained immersed in the respective allergens of the respective capsule 108 ready for use by the user to administer the desired test.

In use, the allergy testing kit 100, as disclosed herein, may be stored in a healthcare provider's office for convenient and rapid retrieval ready for immediate use. To perform an allergy testing procedure, the clinician may remove the allergy testing kit 100 from the sterile packaging and place the capsule 108 in the Base member 102 openings thereof. Then, the user can conveniently grasp the applicator 104 with by means of the grip 114 and pull upwardly thereon to release the applicator 104 from the capsule 108 withdrawing the respective distal ends of the respective prongs from the respective capsule 108s. Because the brush of the respective prongs have been submersed within the respective allergens contained in the capsule 108s, as the respective prongs are withdrawn therefrom, respective drops of the respective allergens are attached to the brush by capillary action. As such, the user is relieved of the time consuming and tedious task of applying allergen solutions individually to the respective prongs and may apply the prongs as a group directly to the patient's skin. This serves to expedite the overall process and contributes significantly to reducing the cost of the treatment. The application of the allergens to the patient is thereby completed in a very short period of time, perhaps in only a matter of seconds.

In one embodiment of the subject matter disclosed herein, the total number of openings in the Base member 102 is seventy two. The openings are organized in a group of eight for use with a respective applicator 104. It is to be appreciated that the applicator 104 may be disposed of after one use, while the containing remaining amounts of the respective plurality of allergens in the respective capsule 108s may be retained for subsequent allergen applications.

As such, a new sterile applicator 104 may be selected and the applicator 104 apparatus stored with such new sterile applicator 104 positioned in covering relationship with the points of the respective prongs thereof submersed in the respective allergens. When the user desires to perform additional allergen testing on another patient, he or she may retrieve the allergy testing kit 100 to repeat the process with the brush of the respective prongs having the allergens pre-applied.

As the application of the respective allergens is repeated, the volume of the allergens contained in the respective capsule 108 will be gradually depleted. It is to be appreciated that the unique shape at the bottom of the respective capsule 108 provide for an efficient use of the respective allergens. As the respective prongs are reintroduced into the wells, the close fitting relationship between the distal ends of the respective prongs to the bottom of the capsule 108 will cause the residual allergens to surround such distal ends such that a sufficient volume of allergen adheres to the respective prongs for subsequent application to the patient. As such, the total volume of expensive allergens to be packaged in the capsule 108s of the allergy testing kit 100 is minimized, reducing costs to the end user while, even as the supply of allergens is being depleted, providing a sufficient volume of the respective allergens pooled in such sinks to be applied to the respective points. As such, the amount of waste allergen material to be disposed of is limited. When the supply of allergens has been depleted, the user may merely discard the relatively inexpensive capsule 108 for disposal with other medical waste material.

From the foregoing, it will be appreciated that the allergy testing apparatus of the present invention provides a convenient device for quick and easy simultaneous application of more than one allergens to a particular patient. The testing kit 100 may be formed of relatively inexpensive thermoplastic materials allowing for convenient disposal of such testing apparatus after its use. The testing apparatus incorporates few components and is of relatively simple construction providing for cost effective and rapid manufacture. The apparatus provides for pre-wetted allergen prongs to eliminate the step of applying respective allergens to each individual respective applicator 104 pick immediately before performing an allergy test resulting in time savings as capsule 108 as conservation of expensive allergen testing materials. In addition, the apparatus provides for efficient use of allergen testing materials thereby further reducing costs while limiting the amount of waste allergen material which must be disposed of.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. An allergy testing kit for applying a plurality of allergens to a cutaneous site on a patient comprising:
a base member having a top side and a bottom side, the base member comprising a plurality of openings,
the top side of the base member comprising a recessed depression concentric to each of the plurality of the openings, wherein the recessed depression having a diameter larger than the opening;
a plurality of capsule configured to be received in each of the plurality of the openings within the base member, the plurality of capsule having a capsule portion, the capsule portion holding a predetermined amount of biologically active allergen,
the plurality of capsule having an opening at the opposite end of the well, the opening having a rim on the outer edge of the opening, where in the rim adapted for insertion into the recessed depression of the base member;
the allergy testing kit having a top cover member configured to fasten with the base member.

2. The allergy testing kit of claim 1 further comprises an applicator having a plurality of circular flat plates (“guards”), a plurality of latitudinal ridges (“beams”) having the guard at each end of the beam, a single upwardly extending latitude rigid (“grip”) adapted for manually grasping the device, the grip attached to the plurality of beams at opposing edge to the guards, and a plurality of downwardly extending prongs each prong connected to a at bottom of the guard.

3. The allergy testing kit of claim 2, wherein each of the plurality of prongs adapted for insertion into the corresponding base member opening.

4. The allergy testing kit of claim 2, wherein the prong comprises a first side and a second side; the first side comprises a brush for capturing liquid from the capsule via capillary attraction, the second side connected to the guard.

5. The allergy testing kit of claim 2, wherein the guard adapted to fit in the base member's recessed depression.

6. The allergy testing kit of claim 1, wherein the each of the prongs having a predetermined fixed length.

7. The allergy testing kit of claim 1 further comprises a plurality of triangular recessed depression to orient an applicator, wherein the apex of the triangle, opposite to the base of the triangle, corresponding to the front end of the applicator.

8. The allergy testing kit of claim 1, wherein the eight of the plurality of openings are grouped in two columns with of four openings in each column.

9. The allergy testing kit of claim 8, wherein a triangular recessed depression is placed between the first two openings.

10. The allergy testing kit of claim 2 further comprises a triangular portion having an apex and a base, the base attached to the front of the outermost beam of the applicator.

11. The allergy testing kit of claim 10, wherein the triangular portion of the applicator having a corresponding triangular recessed depression in the base member, wherein the triangular portion fits into the triangular recessed depression.

12. The allergy testing kit of claim 2, wherein the applicator having four beams with eight prongs.

13. The allergy testing kit of claim 1 further comprises a plurality of capsule shells, wherein each of the plurality of capsule shells configured to receive the capsules containing the biologically active allergen via the opening in the base member.

14. The allergy testing kit of claim 1, wherein the quantity of biologically active allergen suffice for up to thirty allergy tests.

15. The allergy testing kit of claim 2 further comprises enforcement bars attached to the grip.

16. The allergy testing kit of claim 2, wherein the grip is a hollow ridge.

17. The allergy testing kit of claim 1, wherein the top cover member having a first latch placed at the center of the width at the first end and a second latch placed at the center of the width at the second end.

18. The allergy testing kit of claim 1, wherein the base member having a catch mechanics to enable attachment of the top cover member.

19. The allergy testing kit of claim 4, wherein the brush is made with multiple abrading elements.

20. The allergy testing kit of claim 1, wherein each capsule is sealably prepackaged with biologically active allergen having a penantrable screen covering the opening.
